(19) United States
(12) Patent Application Publication
(10) Pub. No.: US 2002/0132076 A1
(43) Pub. Date: Sep. 19, 2002

(54) Reinforced catheter device, catheter stock, and methods and apparatus for making same

(76) Inventor: Robert C. Stevens, Gainesville, FL (US)

Correspondence Address: Michael E. Hudzinski, FAY, SHARPE, FAGAN, MINNICH & McKEE, LLP, 7th Floor, 1100 Superior Avenue, Cleveland, OH, 44114-2518, US

(21) Appl. No.: 10/075,053

(22) Filed: Feb. 13, 2002

Related U.S. Application Data
(63) Continuation-in-part of application No. 09/715,988, filed on Nov. 17, 2000.

(51) Int. Cl. B29D 22/00; B29D 23/00; B65D 1/00; F16L 1/00; B32B 1/08; A61M 25/00

(52) U.S. Cl. 428/35.8; 428/35.9; 604/524

(57) ABSTRACT

A reinforced catheter stock and a reinforced catheter are provided, along with an apparatus and methods for making same. The reinforced catheter stock includes a length of cylindrical tubing and a reinforcement member. During manufacture, the cylindrical tubing is continuously advanced from a pay-out spool to a take-up spool while the reinforcement member is wrapped onto the cylindrical tubing to form the reinforced catheter stock. Thereafter, first and second outer finish coatings are applied to the reinforced catheter stock. The coated catheter stock is cut to desired lengths to form multiple reinforced catheters. A portion of each catheter is ground to provide a soft tip portion having a desired flexibility and finish. One or more marking bands may be disposed near the distal end of each catheter.

CROSS-REFERENCE TO RELATED APPLICATION

This application is a continuation-in-part of U.S. Application Ser. No. 09/715,788, filed Nov. 17, 2000.

### BACKGROUND OF THE INVENTION

The subject invention is directed toward the art of vascular catheters and to catheter manufacturing methods and, more particularly, to intravascular microcatheters of unitary construction provided with an integral continuous coiled wire reinforcement member, and to improved methods and apparatus for manufacturing multiples of such catheters from a continuous feedstock.

Angiographic catheters have been widely used for diagnostic purposes such as in conjunction with the injection of dyes or the like into arteries for the visualization of obstructions, ruptures, or other malformations. Diagnostic catheters are typically constructed with an embedded layered wire braid reinforcement system surrounding the lumen to provide torsional control and to strengthen the catheter body to better withstand high pressure injections.

Catheters of the type described above are shown in my prior U.S. Pat. No. 3,485,234, which issued Dec. 23, 1969. My prior U.S. Pat. No. 3,585,707, which issued Jun. 22, 1971 sets forth generally a method of manufacturing wire braid type angiographic catheters. In addition, my prior U.S. Pat. Nos. 5,738,742 and 5,972,143 describe how to manufacture a plurality of diagnostic catheters having unitary body and tip sections from a continuous feedstock. The teachings of the above prior patents are incorporated herein by reference.

It is likely that wire braid construction will continue to be useful in larger diameter catheters such as in the size range French 8 through French 4. However, in modern medical practice, the use of catheter devices has been broadened to embrace many forms of interventional therapy which require catheters having a smaller diameter. As examples, catheters are presently used in connection with placement of dilation balloons for opening obstructed coronaries and other vessels, for the placement of stints to “prop” open vessels, for introduction of anticoagulants to dissolve clots, and for introduction of coagulants to form clots to “plug” aneurysms or to seal off vessels feeding malignant tumors. The target vessels in the above procedures are typically located in the smaller vessels of the brain, kidney, liver, heart, and other organs. Braided wire catheters, however, are not well suited for applications that require a catheter size of French 3 or smaller. There is a need, therefore, for much smaller catheters that can be extended into the smaller target vessels.

One solution is to construct a catheter that uses a coiled wire reinforcement member within the catheter body as an alternative to the braided wire reinforcement construction scheme. Although the coiled wire construction results in some loss of torsion control, a significantly thinner overall catheter body is enabled. Catheters that include integral coiled wire members have an overall good pushability characteristic and typically do not kink as readily as braided wire construction catheters of the same diameter using the same reinforcement wire diameter.

Another advantage is that coiled reinforcement wire catheters provide a larger lumen size than braided wire type catheters relative to overall catheter body size. Since the reinforcement wire is overlapped in the braided construction as it is braided onto the inner catheter wall construction, the overall reinforcement layer thickness is at least twice as large as in the non-overlapping coiled wire type catheter using the same wire diameter.

U.S. Pat. Nos. 5,733,400 and 5,662,622 teach an intravascular catheter carrying a helical reinforcement member embedded within at least a portion of a tubular wall of the catheter. The catheter body is thin and therefore capable of being advanced into small arteries such as in areas of the brain. However, the catheter taught in the above patents is expensive because it is difficult to manufacture. The catheter body is formed from separate sections which are connected end to end.

More particularly, a prior art manufacturing process includes the steps of joining together multiple tubular catheter reinforcement members in end-to-end, abutting relation. For example, a common arrangement is to join four reinforcement members. Next, a UV curable adhesive is placed on the ends of the joined reinforcement members. The ends are covered with a snug, non-adherent transparent sleeve. The adhesive is UV cured and, thereafter, in the cover sleeve is removed.

One major disadvantage of catheters of the type described above is that the manufacturing method is highly time consuming and labor intensive. Further, the overall catheter assembly is susceptible to failure because it is formed of a plurality of individual parts joined end-to-end.

Accordingly, it is therefore desirable to provide an interventional therapy type catheter having a continuous coil reinforcement member and that is of a substantially unitary construction. Further, it is desirable to provide methods and apparatus for manufacturing multiples of such catheters from a continuous feedstock using a continuous process. In order to reduce manufacturing cycle time, it is desirable to wind the reinforcement wire directly onto an inner substrate layer forming the catheter body in a continuous manner and, thereafter, apply two or more subsequent catheter body layers to produce a large number of catheters from a single feedstock in an efficient manner with minimal labor demands. Individual catheters are simply cut from the feedstock, ground for a selected outer surface finish, and thereafter provided with other finish work as desired.

### SUMMARY OF THE INVENTION

In accordance with one aspect of the present invention, a reinforced catheter is provided. The reinforced catheter comprises an elongate flexible tubular member defining a lumen of the catheter, the tubular member wall having a first end defining a proximal end of the catheter and a second end defining a distal end of the catheter. A continuous coil reinforcement member is carried on the elongate flexible tubular member and extends between the proximal end of the catheter and the distal end of the catheter. First and second continuous flexible coatings cover the coil reinforcement member and the tubular member in an overlapping fashion substantially entirely between the proximal end of the catheter and the distal end of the catheter. The outer coating is harder than the inner coating and is selectively removed over a length of the catheter to provide a thin flexible tip portion of the catheter.

In accordance with another aspect of the present invention, a method of manufacturing multiple reinforced catheters is provided. The method includes providing a selected length of an elongate cylindrical tube carried on opposite first and second spool members with a portion of the cylindrical tube extending between the first and second spool members. A selected length of a reinforcement wire is provided. For substantially the length of the cylindrical tube, the cylindrical tube is advanced from the first spool member to the second spool member while simultaneously reinforcement wire is wrapped onto the portion of the cylindrical tube between the first and second spool members to form a continuous length of reinforced catheter stock. The reinforced catheter stock is then coated with predetermined thicknesses of first and second finish coatings for substantially the length of the cylindrical tube to form a continuous length of coated catheter stock. The coated catheter stock is cut at selected locations corresponding to desired catheter lengths to form a plurality of reinforced catheters. Tip portions of the individual catheters are ground to expose the inner soft finish coating thus forming a flexible tip region.

In accordance with another aspect of the present invention, a reinforced catheter stock for manufacturing reinforced catheters is provided. The catheter stock includes a selected length of an elongate flexible tubular member defining a lumen of the catheter stock, the member having a first end defining a lead end of the catheter stock and a second end defining a trailing end of the catheter stock. A continuous coil reinforcement member is carried on the elongate flexible tubular member and extending between the lead end of the catheter stock and the trailing end of the catheter stock.

In accordance with another aspect of the present invention, a method of manufacturing a reinforced catheter stock is provided. The method includes the steps of providing a selected length of an elongate cylindrical tube carried on opposite first and second spool members with a portion of the cylindrical tube extending between the first and second spool members. A selected length of a reinforcement wire is provided and, while advancing the cylindrical tube from the first spool member to the second spool member, the reinforcement wire is wrapped onto the cylindrical tube at a point between the first and second spool members for substantially the length of the cylindrical tube to form a continuous length of reinforced catheter stock.

In accordance with another aspect of the present invention, an apparatus for manufacturing reinforced catheter stock is provided. The apparatus includes a first support member and a second support member, the first and second support members being spaced apart and carrying an elongate cylindrical tube with a portion of the cylindrical tube extending between the first support member and the second support member. A winder device carries a selected length of a reinforcement member. The winder device is adapted to wind the reinforcement member onto the cylindrical tube at a point between the first and second support members. A control device simultaneously controls i) advancement of the cylindrical tube relative to the winder device and ii) winding the reinforcement member onto said cylindrical tube by the winder device at the point between the first and second support members.

An object of the invention is a reduction in the cost and time associated with manufacturing intravascular catheters with embedded helical coil reinforcement members.

Another object of the invention is the provision of an inexpensive intravascular catheter having a unitary construction that is less prone to failure during use. The catheter of the subject invention includes a continuous embedded helical coil reinforcement member that extends between the proximal and distal ends of the catheter body.

Yet another object of the invention is the provision of a reinforced catheter stock for manufacturing multiple reinforced catheters. The reinforced catheter stock includes a continuous embedded helical coil reinforcement member.

A still further object of the invention is the provision of an apparatus for manufacturing intravascular catheters with embedded helical coil reinforcement members from a continuous feedstock.

Still other objects, advantages, and benefits of the invention will become apparent to those skilled in the art upon a reading and understanding of the following detailed description.

### BRIEF DESCRIPTION OF THE DRAWINGS

The invention may take physical form in certain parts and arrangements of parts, the preferred embodiments of which will be described in detail in this specification and illustrated in the accompanying drawings which form a part hereof, and wherein:

FIG. 1 is a flow chart showing the preferred method for manufacturing reinforced catheter stock and multiple reinforced catheters from a continuous feedstock in accordance with the present invention;

FIGS. 2a-2f are views in side elevation of catheter stock formed in accordance with the present invention from a continuous feedstock shown in various stages of sequential construction;

FIG. 3 is a side elevational view of the coiler tip device for winding the reinforcement wire onto the cylindrical tube as shown in FIGS. 2a-2f;

FIGS. 4a-4d are side elevation views of alternate embodiments of individual catheters formed in accordance with the present invention; and,

FIG. 5 is a diagrammatic view of the preferred apparatus for manufacturing reinforced catheter stock from a continuous feedstock in accordance with the present invention.

### DETAILED DESCRIPTION OF THE PREFERRED EMBODIMENTS

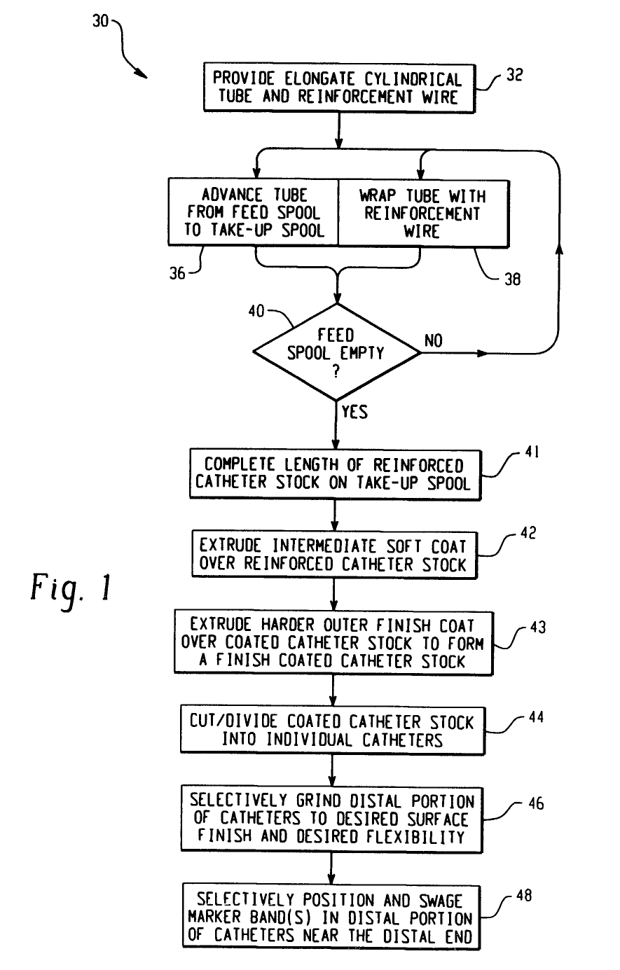

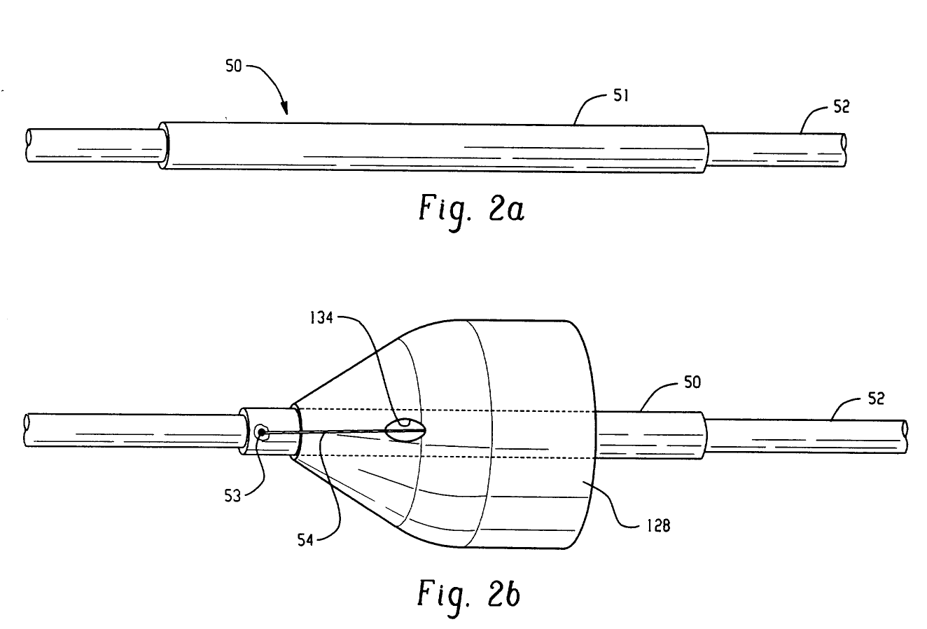

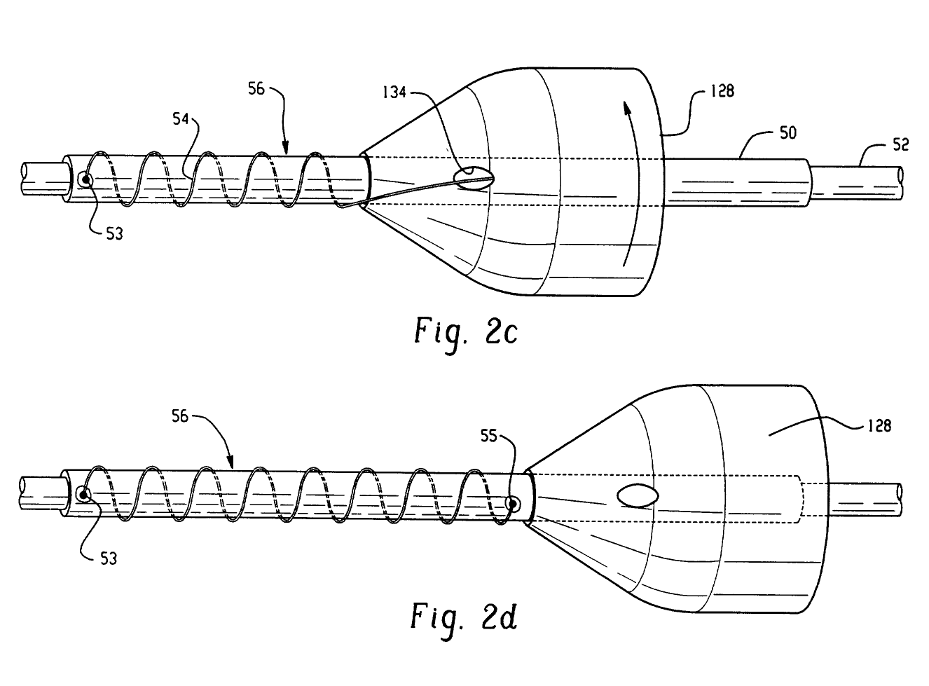

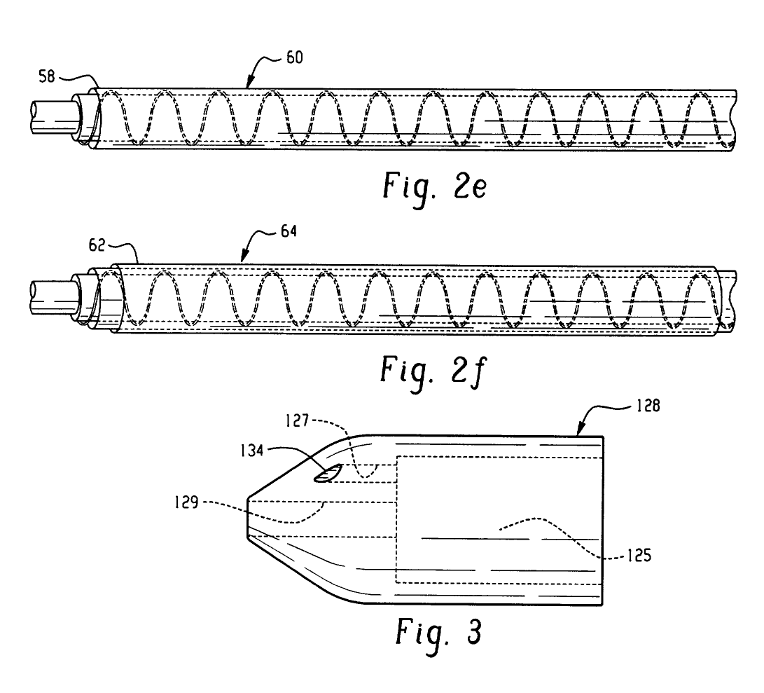

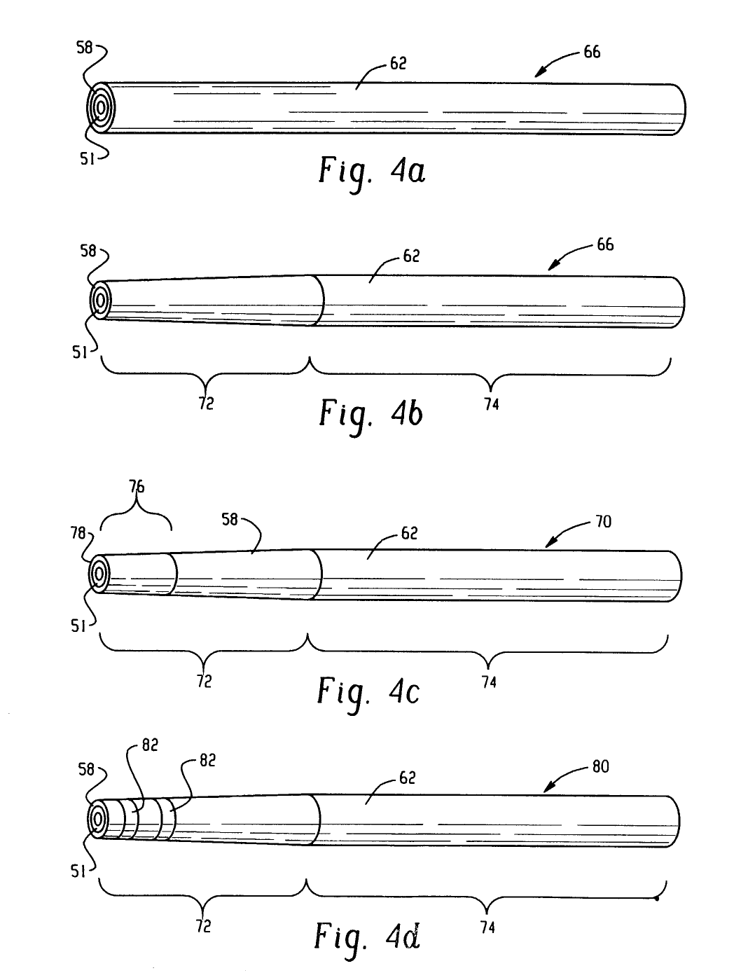

Referring now to the drawings wherein the showings are for the purposes of illustrating the preferred embodiments of the invention only and not for purposes of limiting same, FIG. 1 shows a flow chart illustrating the preferred method 30 of manufacturing a reinforced catheter. Views showing the preferred catheters of the invention in various stages of sequential construction corresponding to a greater or lesser degree with its manufacturing method 30 are set forth in relative diagrammatic form in FIGS. 2a-2f and 4a-4d. It will be noted in comparing FIG. 1 to the prior art manufacturing process described above that the present invention does not require manual attachment of portions of the catheter in an end-to-end abutting relationship. Rather, the entire sequence of steps involves a progressive processing of what is essentially a single element reinforced catheter.

As shown in FIG. 1, the preferred manufacturing method 30 includes the initial step 32 of providing a selected length of an elongate cylindrical tubular member 50. A selected length of a reinforcement wire is also provided. Preferably, tubular member 50 (FIG. 2a) is provided carried on a wire mandrel 52 having an outer diameter that corresponds to the desired lumen diameter of the catheter to be made. As an example, for a French 3 size, the nominal lumen diameter is 0.022 inches. The tube 50 could be formed in many ways, but, in the preferred form of the invention, it is formed by extruding a desired thickness of polytetrafluoroethylene (PTFE) material 51, such as Teflon produced by DuPont, onto a wire mandrel 52 or onto a monofilament mandrel made of a suitable plastic having the desired lumen diameter. In the preferred embodiment illustrated, the mandrel 52 is formed of silver plated copper and is coated with 0.001″ thick PTFE material. Essentially, the mandrel 52 provides support as the catheters are built onto the mandrel, but is removed in a final manufacturing step as will be described below.

The wire mandrel used can have substantially any desired length, but is preferably a substantial number of multiples of the desired final length of the catheter bodies being formed. As an example, it is advantageous to construct multiple catheter tube bodies from a continuous reel of 1,000 feet (30.48 m) or more of mandrel feedstock. For catheters having a nominal length of 59 inches (150 cm), the present invention yields over 200 catheters from a single roll of such feedstock.

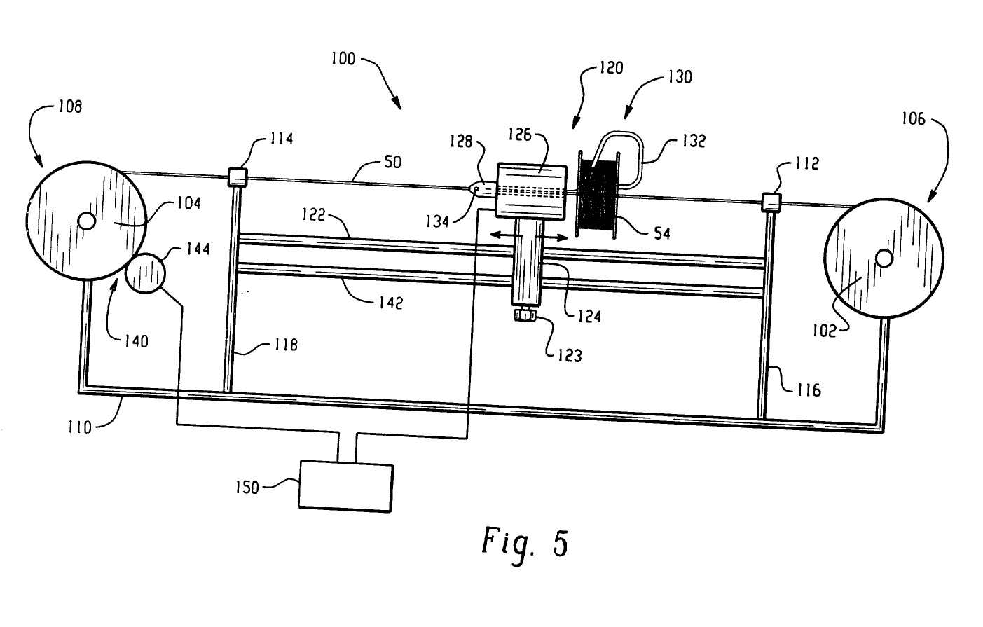

According to the preferred manufacturing method, the entire length, preferably 1,000 feet (30.48 m) of wire mandrel is passed through a conventional extruder to coat the mandrel 52 with a layer of PTFE material 51 having a desired thickness. Preferably the PTFE layer 51 is about 0.001 inches. However, any thickness may be selected as desired. Thereafter, in a wire wrapping step 38, the PTFE tube 50 (FIG. 2a), preferably the entire 1,000 foot (30.48 m) length, with the mandrel 52 in place, is passed through an apparatus 100 (FIG. 5) to be described in detail below for overlaying the PTFE tube 50 with a single strand of a small diameter reinforcement wire 54 to form a reinforced catheter stock 60 (FIG. 2d). Preferably, the reinforcement member is a thin stainless steel wire with a preferred diameter of 0.002 inches. It is to be appreciated, of course, that the tube 50 essentially defines the lumen of the finished catheters and forms the inner body portions of the catheter.

In accordance with the preferred method of the invention and with reference to FIGS. 1 and 5, the wire reinforcement member 54 is wound onto the PTFE tube 50 while the PTFE tube 50 is advanced from a pay-out spool 102 onto a take-up spool 104 in a substantially helical form as shown in FIG. 2c. This enables unwrapped portions of the PTFE tube 50 to be held on large spools on a pay-out end 106 of the winding apparatus shown in FIG. 5 and further, enables wire wrapped portions of the PTFE tube (i.e., reinforced catheter stock) to be collected in a take-up end 108 of the winding apparatus.

Generally, the apparatus 100 includes the pay-out spool 102 and the take-up spool 104 on opposite pay-out and take-up ends 106, 108 of a frame member 110, respectively. A pair of guides 112, 114 are supported on the frame member 110 by respective vertical support members 116, 118, respectively. The first guide 112 is adapted to lead the tube 50 along its path from the pay-out spool 102 to a winder device 120. The second guide 114 is adapted to route the reinforced catheter stock 60 along its path from the winder device 120 to the take-up spool 104. In that way, the tube 50 upstream of the winder and the reinforced catheter stock 60 downstream of the winder are advanced through the guides 112, 114 while the wire reinforcement member 54 is wrapped thereon in steps 36, 38 shown in FIG. 1.

With continued reference to FIG. 5, a winder device 120 is attached at a selected location along a pair of guide beam members 122, 142 of the frame 110. The winder device 120 includes a relatively rigid lower attachment member 124 with a securing mechanism 123 to secure the winder device 120 to the guide beam members 122, 142. Prior to operation, the winder device 120 is slidably moved to a desired position in the horizontal direction shown by the arrows in FIG. 5, and thereafter secured on the guide beam members 122, 142 by tightening the securing mechanism 123. The winder device 120 remains secured at the selected horizontal position during winding operations.

The preferred winder device 120 includes an electric winding motor 126 with a rotatable coiler tip member 128 and wire spool 130 on opposite sides of the winder motor 126. A hollow J-tube member 132 is preferably attached to the coiler tip 128 and supported for rotational movement relative to the wire spool 130 and the winding motor 126. The J-tube member in the arrangement of parts illustrated prevents the coiling wire from becoming tangled in the coiler system. Preferably, the winding motor 126 includes a hollow output drive shaft connected to the coiler tip 128 and J-tube member 132 so that the tubular member 50 carried on the mandrel 52 can be threaded through the winder device 120.

As best shown in FIGS. 2c and 3, a lead end 53 of the wire reinforcement member 54 extends through a hollow body portion 125 of the coiler tip 128, through an offset bore 127 and out from the coiler tip 128 through an offset opening 134. A substantially central bore 129 extends along the longitudinal axis of the coiler tip 128 and is adapted to receive the catheter body 50 therein in a manner shown in FIGS. 2b and 2c. The lead end 53 of the reinforcement member 54 is attached to the lead end of the tube 50 carried on the mandrel 52. An offset opening 134 is provided in the coiler tip 128 as shown to enable the wire reinforcement member 54 to feed off from the wire spool 130 (FIG. 5) and then successively through the J-tube member 132, winding motor 126, and coiler tip 128 substantially as shown. As the winding motor 126 rotates, the wire reinforcement member 54 is payed out from the wire spool 130 and wrapped onto the tubular member 50 while the tube 50 is advanced from the pay-out spool 102 to the take-up spool 104 through the guides 112, 114.

With reference once again to FIG. 1, the preferred method of manufacturing reinforced catheter stock from a continuous feedstock includes the step of advancing the tube 50 carried on the mandrel 52 from the pay-out end 106 of the apparatus 100 toward the take-up end 108. At step 36, the tube is advanced from the pay-out spool 102 and wound onto the take-up spool 104. This presents continuous fresh unwrapped PTFE tube 50 to the winder device 120 from the pay-out spool 102.

In wrapping step 38, the wire reinforcement member 54 is wound onto the tube 50 by simultaneously activating the take-up drive motor 144 and the winding motor 126 using the control device 150. The control device 150 provides coordinated motion between the winding motor 126 and the take-up drive motor 144 which controls translation of the tubular member 50 carried on the mandrel 52 from the pay-out spool 102 onto the take-up spool 104.

The advancement of the tube 50 and wrapping the wire reinforcement member 54 onto the tube (steps 36,38) continues until the entire mandrel coated with tube-forming material is depleted from the pay-out spool 102 at step 40. At this point 41 in the preferred method, a continuous length of reinforced catheter stock 60 is formed and collected on the take-up spool. Preferably, a nominal length of approximately 1,000 feet (30.48 cm.) of reinforced catheter stock 56 is formed.

In step 42, the entire length of the reinforced catheter stock 56 (FIG. 2d) is coated with a predetermined thickness of a continuous soft plastic coating 58 to form a continuous length of coated catheter stock 60 (FIG. 2e). In the preferred embodiment, the coating is a soft plastic material having a Shore hardness of about 40D, such as PEBAX available from Elf Atochem.

In step 43, the entire length of coated catheter stock 60 is coated with a predetermined thickness of a continuous hard plastic coating 62 to form a continuous length of finish coated catheter stock 64 (FIG. 2f). In the preferred embodiment, the finish coating 62 is harder than the base coating 58 and has a shore hardness of about 70D. Preferably, the finish coating is PEBAX available from Elf Atochem.

After the finish coated catheter stock 64 is formed in step 43, the tube is cut or divided in step 44 at selected locations to produce individual reinforced catheters 66 (FIG. 4a) having the length and other properties desired. Using this method, each individual reinforced catheter has an inner wall formed by the PTFE material 51, a wire reinforcement member 54, an intermediate portion formed by a relatively soft, e.g. 40D, material 58, and an outer wall portion formed by the relatively hard, e.g. 70D, finish coating 62.

In the preferred embodiment, the cut catheter lengths are thereafter selectively ground to size and finish at step 46 using a centerless grinding process. Centerless grinders are widely used in industry and in angiographic catheter manufacture in particular. Catheter stock is “fed” though the grinder to remove excess plastic and to bring it to an accurate diameter. The grinder also creates a smooth surface finish. Centerless grinders are also used to grind tapers on catheter tips.

In the centerless grinding step, the part to be ground is rotated under the grinding wheel. The grinding process is preferably accomplished in a manner as described in my earlier U.S. Pat. No. 5,738,742.

The preferred embodiment of the reinforced catheter 68 produced after the grinding step is shown in FIG. 4b. The ground end of the reinforced catheter 68 defines a flexible distal portion 72 and an opposite relatively less flexible proximal portion 74. The distal portion 72 of each catheter 68 is selectively ground to a reduced diameter relative to the proximal portion 74 or main body portion to provide the desired flexibility of the catheter 68 (step 46). The grinding operation is selectively a one of a step grinding operation or a smooth long taper grinding operation.

An example of the preferred embodiment is a microcatheter for use in cerebral procedures. In that example, the overall length of the catheter body including the distal portion 72 together with the proximal portion is 155 cm. long. Preferably, the entire length (155 cm.) of the catheter is ground to a nominal outer diameter of 0.033 in. (72.5 mm). Next, the distal portion 72 of the catheter is ground to a diameter of 0.026 in. which becomes the tip segment. The tip segment can be ground to the desired length relative to the proximal portion 74.

As a preferred example, cerebral microcatheters typically include a soft flexible tip section 72 having an overall length of about 42 cm. It is to be appreciated that a first grinding operation over the length of the catheter essentially forms a catheter body having a substantially uniform cross-sectional dimension. The further grinding step in the distal portion area selectively removes the outer plastic layer 62 leaving exposed the inner more flexible layer 58. This results in a composite catheter having a soft tip with a harder catheter body.

In another preferred embodiment of the invention, a reinforced catheter 70 (FIG. 4c) with a softer tip portion 76 is provided. First, the finish coating 62 is substantially ground away at the tip portion 76 of the catheter 70 exposing the wire reinforcement member 54 on the PTFE tubular member 50. Then, a soft plastic compound 78, for example, Pellethane 55D a urethane product available from Dow Chemical, is fused or molded onto the tip portion 76 of the reinforced catheter 70 (FIG. 4c). The fusing or molding process is preferably accomplished in a manner as described in my earlier U.S. Pat. No. 3,485,234.

Another preferred embodiment includes a reinforced catheter 80 (FIG. 4d) with one or more marker bands as produced in step 48. A first marker band 82 is positioned near the distal end of the catheter 80. The first marker band is secured, preferably by swaging, around the outer surface of the catheter 80. A second marker band 82 is selectively positioned near the distal end of the catheter 80 and spaced apart from the first marker band 82 by about 3.0 cm. The second marker band is also secured, preferably by swaging, around the outer surface of the catheter 80. If desired, the marker bands 82 are selectively positioned at other locations and additional marker bands 82 are added to the reinforced catheter 80 as needed.

The invention has been described with reference to the preferred embodiments. Obviously, modifications and alterations will occur to others upon a reading and understanding of this specification. It is intended to include all such modifications and alterations insofar as they come within the scope of the appended claims or the equivalents thereof.

Having thus described the invention, it is claimed:

1. A reinforced catheter comprising:
an elongate flexible tubular member defining a lumen of the catheter, the tubular member having a first end defining a proximal end of the catheter and a second end defining a distal end of the catheter;
a continuous coil reinforcement member carried on the elongate flexible tubular member and extending between the proximal end of the catheter and the distal end of the catheter;
a first flexible outer coating covering the coil reinforcement member and the tubular member substantially entirely between the proximal end of the catheter and the distal end of the catheter; and,
a second flexible outer coating covering a first portion of the first outer coating between a first transition area of the catheter and said proximal end of the catheter, a second portion of the first outer coating being uncovered by said second outer coating and defining a flexible distal tip of said catheter, the first coating being softer than said second coating.

2. The reinforced catheter according to claim 1 wherein the first flexible outer coating is softer than said second flexible outer coating.

3. The reinforced catheter according to claim 2 wherein:
said first flexible outer coating has a Shore hardness of about 40D; and, said second flexible outer coating has a Shore hardness of about 70D.

4. The reinforced catheter according to claim 1, further comprising a marker band disposed adjacent the distal end of the catheter on the outer coating.

5. The reinforced catheter according to claim 4, wherein the marker band is formed of a one of gold material and platinum material.

6. The reinforced catheter according to claim 1, wherein the elongate flexible tubular member is formed of a polytetrafluoroethylene (PTFE) material.

7. The reinforced catheter according to claim 1, wherein the continuous coil reinforcement member is a stainless steel wire.

8. The reinforced catheter according to claim 1, wherein the continuous coil reinforcement member defines a helical pattern.

9. The reinforced catheter according to claim 1, wherein a thickness of the distal end of the catheter is less than a thickness of the proximal end of the catheter.

10. The reinforced catheter according to claim 1, wherein the first outer coating is comprised of one of a group of materials consisting of nylon material and urethane material.

11. The reinforced catheter according to claim 1, wherein the second outer coating is comprised of a nylon material.

12. A method of manufacturing multiple reinforced catheters comprising the steps of:
providing a selected length of an elongate cylindrical tube carried on opposite first and second spool members with a portion of the cylindrical tube extending between the first and second spool members;
providing a selected length of a reinforcement wire;
for substantially the length of the cylindrical tube, advancing the cylindrical tube from the first spool member to the second spool member while simultaneously wrapping the reinforcement wire onto said portion of the cylindrical tube between the first and second spool members to form a continuous length of reinforced catheter stock;
coating the reinforced catheter stock with a predetermined thickness of a first coating and followed by a second coating harder than said first coating for substantially the length of the cylindrical tube to form a continuous length of coated catheter stock; and,
cutting the coated catheter stock at selected locations corresponding to desired catheter lengths to form a plurality of reinforced catheters.

13. The method of manufacturing multiple reinforced catheters according to claim 12 further including the step of grinding the second coating of any one or more of said plurality of reinforced catheters to expose a portion of the first coating and to provide a desired outer surface finish and a desired flexibility along the longitudinal length of the catheter.

14. The method of manufacturing multiple reinforced catheters according to claim 13 further including the step of swaging a marker band around the outer surface of the coating at a distal end of the any one or more of said plurality of reinforced catheters.

15. The method of manufacturing multiple reinforced catheters according to claim 14, wherein the step of swaging the marker band includes swaging a marker band formed of one of a group of materials consisting of gold and platinum.

16. The method of manufacturing multiple reinforced catheters according to claim 14, wherein the grinding step includes grinding a portion of the catheter beginning at a first end defining a distal end of the catheter for a predetermined distance along the longitudinal length of the catheter toward a second end defining a proximate end of the catheter.

17. The method of manufacturing multiple reinforced catheters according to claim 16, wherein the grinding step includes grinding the portion of the catheter such that the thickness of the finish coating at the distal end of the catheter is less than the thickness of the finish coating at the proximate end of the catheter.

18. The method of manufacturing multiple reinforced catheters according to claim 17, further including the step of coating a ground portion of the catheter with a predetermined thickness of a soft finish coating.

19. The method of manufacturing multiple reinforced catheters according to claim 18, wherein the step of coating the ground portion with said soft finish coating includes coating the ground portion with a urethane material.

20. The method of manufacturing multiple reinforced catheters according to claim 12, wherein the cylindrical tube is a polytetrafluoroethylene (PTFE) material.

21. The method of manufacturing multiple reinforced catheters according to claim 12, wherein the reinforcement wire is a stainless steel wire.

22. The method of manufacturing multiple reinforced catheters according to claim 12, wherein the wrapping step includes wrapping said reinforcement wire onto said cylindrical tube in a helical pattern.

23. The method of manufacturing multiple reinforced catheters according to claim 12, wherein the coating step includes coating the reinforced catheter stock with a predetermined thickness of said first coating followed by a predetermined thickness of said second coating, the first coating having a Shore hardness of about 40D and said second coating having a Shore hardness of about 70D.

24. A reinforced catheter stock for manufacturing reinforced catheters, the catheter stock comprising:
a selected length of an elongate flexible tubular member defining a lumen of the catheter stock, the tubular member having a first end defining a lead end of the catheter stock and a second end defining a trailing end of the catheter stock; and
a continuous coil reinforcement member carried on the elongate flexible tubular member and extending between the lead end of the catheter stock and the trailing end of the catheter stock.

25. The reinforced catheter stock according to claim 24, further comprising a continuous outer coating of first and second materials covering the coil reinforcement member and the tubular member substantially entirely between said lead end of the catheter stock and the trailing end of the catheter stock.

26. The reinforced catheter stock according to claim 25, wherein:
the continuous coil reinforcement member defines a helical pattern;
the first material has a Shore hardness of about 40D; and,
the second material has a Shore hardness of about 70D.

27. The reinforced catheter stock according to claim 24, wherein the elongate flexible tubular member is a polytetrafluoroethylene (PTFE) material.

28. The reinforced catheter stock according to claim 24, wherein the continuous coil reinforcement member is a stainless steel wire.

29. A method of manufacturing a reinforced catheter stock, the method comprising the steps of:
providing a selected length of an elongate cylindrical tube carried on opposite first and second spool members with a portion of the cylindrical tube extending between the first and second spool members;
providing a selected length of a reinforcement wire; and
while advancing the cylindrical tube from the first spool member to the second spool member, wrapping the reinforcement wire onto the cylindrical tube at a point between the first and second spool members for substantially the length of the cylindrical tube to form a continuous length of reinforced catheter stock.

30. The method of manufacturing reinforced catheter stock according to claim 27, further comprising the step of coating the reinforced catheter stock with a predetermined thickness of a first finish coating then a second finish coating harder than said first finish coating for substantially the length of the cylindrical tube to form a continuous length of coated catheter stock.

31. The method of manufacturing reinforced catheter stock according to claim 29, wherein the step of providing said elongate cylindrical tube includes providing a polytetrafluoroethylene (PTFE) material.

32. The method of manufacturing reinforced catheter stock according to claim 29, wherein the step of providing said selected length of said reinforcement wire includes providing stainless steel wire.

33. The method of manufacturing reinforced catheter stock according to claim 29, wherein the wrapping step includes wrapping said reinforcement wire onto said cylindrical tube in a helical form.

34. An apparatus for manufacturing reinforced catheter stock, the apparatus comprising:
a first support member and a second support member, the first and second support members being spaced apart and carrying an elongate cylindrical tube with a portion of the cylindrical tube extending between the first support member and the second support member;
a winder device carrying a selected length of a reinforcement member, the winder device being adapted to wind the reinforcement member onto the cylindrical tube at a point between the first and second support members; and,
a control device simultaneously controlling i) advancement of the cylindrical tube relative to the winder device and ii) winding the reinforcement member onto said cylindrical tube by the winder device at the point between the first and second support members.

35. The apparatus according to claim 34, wherein said first support member includes a pay-out spool and said second support member includes a take-up spool, the pay-out spool and the take-up spool being responsive to the control device to pay out the elongate cylindrical tube from the pay-out spool and onto the take-up spool.

36. The apparatus according to claim 34, wherein the elongate cylindrical tube is a polytetrafluoroethylene (PTFE) material.

37. The apparatus according to claim 34, wherein the winder device includes:
a coiler tip member defining i) a central bore adapted to receive said cylindrical tube at the point between the pair of spaced apart support members, and ii) an offset opening carrying said reinforcement member, the coiler tip member being selectively rotatable relative to said cylindrical tube to wind the reinforcement member onto the cylindrical tube at selected varied angles relative to a plane perpendicular to a longitudinal axis of the cylindrical tube.

38. The apparatus according to claim 37, wherein the winder device further includes:
a motor for rotating the coiler tip member relative to the cylindrical tube;
a spool for carrying the reinforcement member; and,
a tubular member adapted to rotate with the coiler tip member to feed the reinforcement member from said spool and through the offset opening of the coiler tip member as the reinforcement member is wound onto the cylindrical tube.

39. The apparatus according to claim 38, wherein the winder device is adapted to wind the reinforcement member onto the cylindrical tube in a helical pattern.

40. The apparatus according to claim 34, wherein the reinforcement member is comprised of a stainless steel wire.
